(19) United States
(12) Patent Application Publication
(10) Pub. No.: US 2019/0106369 A1
(43) Pub. Date: Apr. 11, 2019

(54) Processing Base-Catalysed Alkoxylation Products

(71) Applicant: Evonik Degussa GmbH, Essen (DE)

(72) Inventors: Frank Schubert, Neukirchen-Vluyn (DE); Wilfried Knott, Essen (DE); Andrea Villa, Bottrop (DE); Volker Zellmer, Bottrop (DE); Gerrit Wienhoefer, Chesterfield, VA (US)

(73) Assignee: Evonik Degussa GmbH, Essen (DE)

(21) Appl. No.: 16/087,762

(22) PCT Filed: Mar. 6, 2017
(86) PCT No.: PCT/EP2017/055141

§ 371 Date: Sep. 24, 2018

(30) Foreign Application Priority Data
Apr. 4, 2016 (EP) 16163614.7

(51) Int. Cl. C07C 41/36; C08G 65/30; B01J 39/20; B01J 39/05

(52) U.S. Cl. CPC C07C 41/36; B01J 39/05; B01J 39/20; C08G 65/30

(57) ABSTRACT

A method of processing of base-catalysed alkoxylation products using sulphonic acid-containing ion exchangers is described, comprising providing a mixture comprising the alkali-catalysed alkoxylation product to be processed, alcohol having 1 to 4 carbon atoms and water, treating this mixture with a sulphonic acid-containing cation exchanger at >40° C. and removal of the alkoxylation product from the mixture thus treated.

### FIELD

The present invention relates to a method of processing alkali-catalysed alkoxylation products using sulphonic acid-containing ion exchangers. The invention further relates to the thus obtainable alkoxylation products and to the use thereof for producing alkali-metal-free and low odour descendent products such as silicone polyethers and surfactants.

### BACKGROUND

Alkaline catalysts such as alkali metal hydroxides and alkali metal alkoxides are widely used in alkoxylation reactions. This comprises addition of alkylene oxides such as ethylene oxide and propylene oxide onto typically hydroxyl- or carboxyl-functional starting compounds such as alcohols, phenols or carboxylic acids under strongly basic conditions. The alkoxylation products obtained, often referred to as polyethers, polyetherols or polyether polyols, in their crude state comprise residues of the alkaline catalyst and must in most cases be worked up in a downstream process step prior to application, i.e. neutralized, freed from alkaline and salt residues, and filtered.

### SUMMARY

Neutralization is often achieved by addition of aqueous phosphoric acid or sulphuric acid. The catalyst residues are initially converted into alkali metal phosphates, alkali metal hydrogenphosphates, alkali metal sulphates or alkali metal hydrogensulphates, precipitated after distillative removal of water and subsequently removed by filtration. The removal of alkali metal salts is often a time-consuming and quality-determining step. The salt removal achieved is generally not quantitative since a portion remains dissolved in the polyether and another portion is in such a finely crystalline state that it cannot be removed completely from the end product with reasonable technical means, even using filtration aids. Salt residues tainted with polyether remain in the production reactor after the neutralization and in batch operation need to be dissolved/rinsed out before commencement of the next production. A wastewater contaminated with organic and salt residues is thus generated. The waste product obtained is a damp, polyether-comprising filtercake which requires disposal and results in a loss in yield.

### DETAILED DESCRIPTION

Depending on the chemical makeup of the polyether, the neutralization of the alkaline alkoxylates with carboxylic acids such as acetic acid or lactic acid often results in soluble alkali metal carboxylates which cannot be removed by precipitation and filtration. While this avoids a number of the abovementioned processing steps and disadvantages, the alkali metal carboxylates dissolved in the end product are undesired byproducts for many applications. Accordingly, carboxylate proportions have a disruptive effect on subsequent reactions of the neutralized polyether. Platinum-catalysed hydrosilylation reactions of hydrosiloxanes with terminally unsaturated polyethers such as allyl polyethers often give rise to catalyst poisons which inhibit the Pt catalyst. While the alkali metal carboxylates are dissolved in the polyether, further chemical processing, for example modification of the polyether with hydrophobic structural units such as siloxanes or hydrocarbon radicals, causes said carboxylates to precipitate out of the reaction product formed and to cause unacceptable haze. The high viscosity of the descendent products often renders a subsequent filtration of the disruptive salt residues impossible and salts should therefore be removed directly from the alkoxylation products and before the further processing.

Polyethers are very versatile compounds. An important class of descendent products are polyether-siloxane copolymers, also known as polyether siloxanes, polyether silicones or silicone polyethers. The broad applicability stems from the ability to achieve targeted adjustment of numerous operating principles by suitable combination of siloxane and polyether structures. Of particular importance are polyethers derived from allyl alcohol which are reacted with Si—H-functional siloxanes in the presence of Pt catalysts to afford SiC-bonded polyether siloxanes. The alkali-catalysed production of allyl polyethers unavoidably results in isomerization of a portion of the allyl groups to afford thermodynamically more stable propenyl groups. The hydrosilylation reaction requires terminal double bonds for the Si—C bond forming reaction and the propenyl polyethers formed are therefore unreactive byproducts in the context of the polyether siloxane synthesis. This is dealt with by employing a considerable excess of the polyether component in the hydrosilylation to ensure a quantitative Si—H conversion.

As is disclosed in DE 10024313 A1 the presence of propenyl polyethers causes various further undesired properties. Under the influence of (atmospheric) humidity and promoted by traces of acid propenyl polyethers undergo hydrolysis. Propionaldehyde is liberated over time and partly outgassed. Cyclic oligomers (aldoxane, trioxane) and also acetals which have a tendency for retrocleavage and thus for renewed aldehyde liberation are formed from propionaldehyde in secondary reactions. Especially products employed in the personal care sector and in interiors require odour neutrality and thus often an aftertreatment. Acetals are often formed even during polyether production by reaction of aldehyde with the OH-functional polyether. Acetals increase viscosity via increased molar mass and skew the desired properties of the end products.

The prior art describes various methods for avoiding or remedying the recited problems for allyl polyether-based systems:

EP 0118824 A1 describes polyether siloxanes as oils for cosmetic purposes having a total content of carbonyl-bearing compounds (aldehydes and ketones) of ≤100 ppm which are obtained by hydrosilylation in the presence of antioxidants and optionally a buffer.

JPH 07304627 A discloses a method of treatment of allyl polyether-based polyether siloxanes with aqueous HCl at 60° C. over 24 h. An acid-induced hydrolysis of propenyl polyether proportions with removal of propionaldehyde is also described in J. Soc. Cosmet. Japan (1993), 27(3), 297-303. EP 0398684 A2 describes the production of low-odour silicone polyethers by treatment with dilute hydrochloric acid for several hours at elevated temperature with subsequent vacuum distillation to obtain a virtually odourless copolymer.

According to U.S. Pat. No. 4,515,979 the addition of phytic acid likewise results in a reduction in undesired odours in polyether siloxanes based on allyl polyethers. The disadvantage is that the phytic acid remains in the end product thus preventing use in sensitive sectors such as in paints and personal care products. Processes such as catalytic pressure hydrogenation are complex and costly and thus acceptable only for small high-value fields of application.

As disclosed in EP 1531331 A2 the polyether siloxanes treated with acid as per the prior art processes are unsuitable for use as polyurethane foam stabilizers. Acid treatment has disastrous effects on performance and instead of the desired foam stabilization a collapse of the labile foam structure is observed, particularly in flexible foam systems. Instead, a mild treatment of the silicone polyethers with hydrogen peroxide followed by a distillative removal of odour-forming additions is preferred.

The prior art is familiar with alternative alkoxylation catalysts which make it possible to obtain salt-free, virtually propenyl-free and olfactorily favourable polyethers. These include double metal cyanide (DMC) catalysts, as reported for example in EP 2241352 A2. As is known to one skilled in the art DMC catalysts result in polyethers having a very narrow molar mass distribution on account of their completely different mechanism of action. The sequence of ethyleneoxy and propyleneoxy units for mixed polyethers in statistically mixed alkoxylates differs from said sequence in alkali-catalysed polyethers. Both factors influence product properties such as hydrophilicity/hydrophobicity, haze point or compatibility in various media. The use of DMC catalysis is further subject to certain restrictions. Especially the allyl and butyl polyethers important for polyether siloxanes cannot be produced by the direct route using DMC catalyses since for example short-chain alcohols inhibit the DMC catalyst. Accordingly for many applications DMC catalysis does not represent a useful alternative to the widespread alkaline catalysis.

DE 10024313 A1 discloses a method in which a cation exchanger is employed to remove alkali metal ions from alkaline alkoxylates and to avoid incorporation of phosphate into the end product. The alkaline alkoxylation product is dissolved in an inert organic solvent, treated at 20-60° C. with a cation exchanger and lastly freed of solvent.

U.S. Pat. No. 5,342,541 discloses the use of acid cation exchangers with the aim of reducing the content of propenyl polyethers in the end product. The disadvantage of the method is the incorporation of traces of acid from the employed gel-type ion exchangers into the polyether treated therewith, which renders the direct use of the products in polyurethanes practically impossible. The method therefore requires an aftertreatment of the acidic polyethers with an epoxy compound as an acid scavenger. The applicability of this process is limited to gel-type ion exchangers since only these have pores small enough to ensure that long-chain polymers are not admitted. The avoidance of direct contact with the acidic sulphonic acid groups suppresses degradation of the polyether.

The present invention accordingly has for its object the provision of a mild, environmentally-friendly and efficient method of purifying alkali-catalysed alkoxylation products and also the provision of correspondingly purified alkoxylation products.

It has been found that, surprisingly, high quality and versatile purified polyethers are obtained when the alkali-catalysed crude products are treated in alcoholic-aqueous solutions at elevated temperatures of more than 40° C. with sulphonic acid ion exchangers, preferably with specially selected macroporous sulphonic acid-containing ion exchangers.

The present invention accordingly provides a method of processing alkali-catalysed alkoxylation products using sulphonic acid-containing ion exchangers, comprising
a) providing a mixture comprising the alkali-catalysed alkoxylation product to be processed, alcohol having 1 to 4 carbon atoms and water,
b) treating the mixture obtained from step a) with a sulphonic acid-containing cation exchanger at >40° C.,
c) removal, preferably distillative removal, of the alkoxylation product from the mixture obtained in step b).

In the context of the present invention the terms “alkoxylate” and “alkoxylation product” are used synonymously and comprehend in particular the reaction products formed by alkali-catalysed polyaddition of alkylene oxides onto hydroxyl groups and/or carboxyl groups, also known as polyethers, polyols, polyetherols, polyethylene glycols or polypropylene glycols. This includes pure substances and also mixtures obtained using different alkylene oxides and/or different hydroxyl- and/or carboxyl-bearing starting compounds.

The subject matter of the invention makes it possible not only to remove alkali metal ions/alkali metals from the alkaline alkoxylates and neutralize the alkoxylates but also to remove undesired odour-forming compounds or additions such as propenyl polyethers or acetals and thus to ensure a route to practically salt-free and olfactorily favourable, versatile polyethers.

The combination of solvent mixture, comprising alcohol and water, and preferably short contact times at elevated temperatures (T>40° C.) on sulphonic acid-containing ion exchangers, preferably having specially selected pore sizes, allows desalting and elimination of odour-forming ingredients in but a single process.

It is made possible to provide purified and practically salt-free alkoxylation products having at most a low residual acid content.

It is made possible to provide purified polyethers having a reduced content of odour-forming additions, for example of propenyl ethers, aldehydes and acetals, and said polyethers therefore require no further aftertreatment and may be employed directly for producing descendent products.

The purified polyethers produced according to the invention combine the broader molar mass distribution important for some applications and typical for alkali-catalysed polyethers with the advantages of the generally salt- and propenyl-free DMC-catalysed polyethers.

The present invention accordingly makes possible the use of the alkoxylation products obtainable in accordance with the invention in the production of PUR foam, polymers such as polyether siloxanes and polyesters, as polyurethane foam stabilizers, in paints, coatings, adhesives and sealants, binders, cosmetic preparations, personal care products and cleaning products, as surfactants, emulsifiers, dispersants, defoamers, wetting agents, friction reducers, lubricants, glidants, release agents, additives in fuels such as petrol and diesel and rheology modifiers and the provision of descendent products of particularly high quality, notable for example for particular odour neutrality.

The terms “alkali-metal-free” and “salt-free” are to be understood in the context of the present invention as meaning that preferably less than 10 ppm, in particular less than 5 ppm, of alkali metals are present.

In a preferred embodiment of the invention the method according to the invention is used for removal of alkali metal residues and odour-forming additions from the alkali-catalysed alkoxylation products.

Preferred implementation of step a) of the method according to the invention:

The alkoxylation products employed in step a) are alkali-catalysed alkoxylation products. These are known per se to one skilled in the art. Said products may be produced by the methods known in the prior art in the presence of alkali metal hydroxide or alkali metal alkoxide catalysts and normally comprise 100 ppm to 6000 ppm, preferably 500 ppm to 4000 ppm, of alkali metals.

Widespread products are for example alkali-catalysed alkoxylates that have been synthesized using sodium hydroxide, potassium hydroxide, sodium methoxide and/or potassium methoxide. Such alkali-catalysed alkoxylates may be employed with preference in the context of the present invention.

The method according to the invention is applicable to alkaline alkoxylation products of any desired molar mass. Preference is given to alkoxylation products having weight-average molar masses Mw of 150 g/mol to 15 000 g/mol, preferably 200 g/mol to 10 000 g/mol, particularly preferably 400 g/mol to 5000 g/mol. The weight-average molar masses Mw are determinable by GPC: SDV 1000/10 000 A column combination (length 65 cm), temperature 30° C., THF as mobile phase, flow rate 1 ml/min, sample concentration 10 g/1, RI detector, evaluation against polypropylene glycol standard.

The polydispersity of the employed alkoxylation products may be varied within wide limits. Preferably employed alkaline alkoxylates have a polydispersity Mw/Mn of 1.04 to 1.5, particularly preferably between 1.05 and 1.35, as per GPC using a PPG standard.

In a particularly preferred embodiment of the invention the alkali-catalysed alkoxylation products to be processed originate from an alkali-metal-hydroxide- and/or alkali-metal-alkoxide-catalysed alkoxylation process, have a molar mass Mw (GPC using PPG standard) of 150 g/mol to 15 000 g/mol, preferably 200 g/mol to 10 000 g/mol, particularly preferably 400 g/mol to 5000 g/mol and have a polydispersity of 1.04 to 1.5, particularly preferably between 1.05 and 1.35.

Both alkoxylation products liquid at room temperature (20° C.) and alkoxylation products solid at room temperature are employable since these are added to a solvent mixture before the ion exchanger treatment. The viscosity of the resulting mixture may be adjusted via the amount of solvent.

The alkali-catalysed alkoxylates employed in step a) are in particular the reaction products of a polyaddition of epoxy compounds onto an OH-functional or carboxyl-functional starting compound. Preferably employed alkylene oxides are ethylene oxide, propylene oxide, 1-butylene oxide, 2-butylene oxide, isobutylene oxide and styrene oxide, ethylene oxide and propylene oxide being particularly preferably employed. The epoxy monomers may be employed in pure form, successively or in admixture. The polyoxyalkylenes formed are thus subject to a statistical distribution in the end product. The correlations between metered addition and product structure are known to those skilled in the art.

Suitable OH-functional starters are in principle all saturated or unsaturated, linear or branched, mono- or polyhydric OH-functional starting compounds. Preferred starters are compounds from the group comprising alcohols, diols, polyols, polyetherols and phenols, preferably allyl alcohol, n-butanol, 1-octanol, 1-decanol, 1-dodecanol, fatty alcohols having 8-22 carbon atoms in general such as stearyl alcohol, 2-ethylhexanol, isononanol, 3,5,5-trimethylhexanol, cyclohexanol, benzyl alcohol, 1,2-hexanediol, 1,6-hexanediol, 1,4-butanediol, neopentyl glycol, hexylene glycol, eugenol, alkylphenols, cashew nut shell liquid, hexenol, ethylene glycol, propylene glycol, di-, tri- and polyethylene glycol, 1,2-propyleneglycol, di- and polypropylene glycol, trimethylolpropane, glycerol, polyglycerol, pentaerythritol, sorbitol and hydroxyl-bearing compounds derived from natural products.

Preferred starting compounds have on average 1 to 6, preferably 1 to 3, particularly preferably 1 to 2, very particularly preferably 1, OH group(s) per molecule.

Accordingly in a preferred embodiment of the invention the alkali-catalysed alkoxylation products to be processed have 1 to 6 OH groups, preferably 1 to 3 OH groups, particularly preferably 1 to 2 OH groups, in particular 1 OH group.

Furthermore, any desired carboxylic acids may be employed as starters. Preference is given to mono- or polyfunctional aliphatic carboxylic acids, aromatic carboxylic acids and cycloaliphatic carboxylic acids. Especially preferred are aliphatic, saturated or unsaturated, linear or branched carboxylic acids having 6 to 22 carbon atoms, for example decanoic acid, undecanoic acid, dodecanoic acid, octadecanoic acid, 2-ethylhexanoic acid, isononanoic acid, 3,5,5-trimethylhexanoic acid, neodecanoic acid, isotridecanoic acid, isostearic acid, undecylenic acid, oleic acid, linoleic acid and ricinoleic acid. Likewise preferred are aromatic carboxylic acids such as benzoic acid and cinnamic acid.

Very particular preference is given to using allyl polyethers since for these products the utility of the method according to the invention in the form of extensive decomposition of propenyl polyethers present therein is particularly pronounced.

According to the invention the alkaline alkoxylation product is mixed with alcohol having 1 to 4 carbon atoms and water. Suitable alcohols are methanol, ethanol, 1-propanol, isopropanol, 1-butanol, 2-butanol and isobutanol, methanol and ethanol being used with preference. Water is added as a further solvent component. The ratio of alcohol to water may be varied within wide limits and is adapted to the polyether structure and thus to the solubility of the alkoxylation product to be purified in each case. In the method according to the invention the ratio of alkoxylation product to alcohol and water is preferably chosen such that a homogeneous, ideally clear solution is formed.

To enhance economy and to avoid waste (recycling) the alcohol/water distillate recovered in step c) may be reused for producing the alkoxylate solution in step a). Pure alcoholic solvent and/or water may be added to this distillate as required to establish the required solvent composition.

The mixture, in particular solution, for treatment with the ion exchanger is advantageously composed to an extent of 35 to 95 wt %, preferably 45 to 85 wt %, particularly preferably 50 to 80 wt %, of the alkaline alkoxylation product. In the mixture, preferably solution, the proportion of the alcohol is advantageously 4 to 64 wt %, preferably 12 to 53 wt %, particularly preferably 17 to 48 wt %. The water content of the solution is preferably 1 to 15 wt %, preferably 2 to 10 wt %, particularly preferably 3 to 8 wt %. The proportions of alkoxylation products, alcohol and water sum to 100 wt % provided that no further substances are present.

Preferred implementation of step b) of the method according to the invention:

In the context of the method according to the invention all known sulphonic acid-containing cationic exchangers may be employed. Synthetic-resin-based cation exchangers having sulphonic acid groups, for example sulphonated styrene-divinylbenzene polymers, have proven particularly effective in the polyether purification according to the invention. It was found that, surprisingly, macroporous sulphonic acid-containing cation exchangers are particularly suitable for the purposes of the present invention. Even short contact times of preferably less than 50 minutes, under preferred conditions less than 40 minutes, are sufficient to remove alkali metal ions and to bring about for example the hydrolysis of propenyl polyethers and acetals.

Numerous sulphonic acid-containing cation exchangers are commercially available on the market. These include ion exchangers from DOW (trade names for example Amberlyst®, Amberjet® and Amberlite®), Lanxess (trade name Lewatit®) and Purolite (Purolite®).

Particularly suitable for the method according to the invention are granular macroporous ion exchangers having sulphonic acid groups. Preferred ion exchangers advantageously comprise particles having an average particle size of 500-900 μm, measured by sieve analysis and an ion exchange capacity of not less than 1.5 equ./litre (H⁺ form) which corresponds to a preferred embodiment of the invention. These include for example the cation exchangers Amberlyst® 15 and Amberlite® 252H.

Immediately employable macroporous and water-containing sulphonated ion exchangers are for example those already present in H-form from the factory. These may be employed without pretreatment. After use the preferred ion exchangers completely or partly laden with alkali metal ions may be regenerated in known fashion with strong aqueous acids such as sulphuric acid or hydrochloric acid, i.e. converted back into the H-form and reused many times.

The treatment of the alkoxylate solution from step a) with the abovementioned sulphonic acid-containing cation exchangers may be effected either in a batch process or else continuously and in a stirred reactor or in a fixed-bed process.

In the case of batch operation in a stirrable container the alcoholic-aqueous alkoxylate solution from step a) and the sulphonic acid-containing ion exchanger may be initially charged in H-form and brought to the desired temperature. The amount of ion exchanger employed depends on the alkali metal content of the alkoxylate solution and the available capacity (content of usable SO₃H groups) in the ion exchanger. To achieve quantitative removal of alkali metals from the alkoxylate an at least stoichiometric amount of sulphonic acid groups of the ion exchanger based on the alkali metal ions to be removed must be employed. Preference is given to using an amount of ion exchanger corresponding to an at least 0.1 molar excess of acid groups based on the alkali metal concentration to be removed. A greater ion exchanger excess is not detrimental but on the contrary is rather conducive to a rapid and thorough purification of the alkoxylation products. The progress of the processing is most easily monitored via a submerged pH probe. The mixture of ion exchanger and alkoxylate solution is in particular stirred until the initial pH of 12 to 14 has fallen to not more than pH 7.

The thus obtained alkali-metal-free solution preferably has a residual content of alkali metal based on the purified alkoxylation product of less than 10 ppm, preferably of less than 5 ppm.

The temperature influences the duration of the neutralization and the simultaneously conducted hydrolysis of any propenyl polyethers and acetals present and is greater than 40° C., preferably greater than 60° C., particularly preferably greater than 70° C. In open systems the maximum temperature is limited only by the boiling point of the alkoxylate/alcohol/water mixture and the method according to the invention may also be conducted at boiling point and under reflux. In a closed pressurized stirred reactor the treatment of the alkoxylate solution with the ion exchanger may also be performed above the boiling point under pressure, for example at 100° C. in ethanol/water.

In a preferred embodiment of the invention, in step b) the mixture from step a) is passed through an ion exchanger bed at 45° C. to 100° C., preferably more than 60° C. to 100° C., particularly preferably more than 70° C. to 100° C.

The temperature, the type and amount of alcoholic solvent, the water content, the alkali metal content and the chemical makeup of the alkoxylation product to be purified and also the usage amount of the ion exchanger influence the duration of the purification. The duration of the processing is defined as the time measured from addition of the ion exchanger to the alkaline alkoxylate solution until achievement of a pH of 7. In a preferred embodiment the influencing factors are chosen such that the pH of 7 is achieved within less than 50 minutes. Short residence times of less than 40 minutes and in particular of less than 25 minutes until achievement of a pH of 7 are particularly preferred.

The use of more solvent, more ion exchanger and higher temperatures generally brings about an acceleration of the ion exchange and of the purification.

Passing the alkaline solution from step a) through a vessel filled with sulphonic acid-containing ion exchanger at >40° C. represents an advantageous and easy-to-implement alternative to the stirred reactor process. Here, the alkali-metal-comprising alkoxylate/alcohol/water mixture, preferably solution, is passed continuously through the temperature-controllable ion exchanger fixed bed, e.g. with the aid of a pump. The ion exchange fixed bed is preferably located in a column or in a tube which may be externally temperature-controlled. Thus, double-shelled vessels where a liquid heat transfer medium can circulate in the outer shell are particularly suitable. Connected to an external, controllable heat transfer plant and provided with a temperature measuring point in the interior of the vessel, the temperature in the fixed bed may be adjusted to a predetermined value and kept constant over the entire period of operation.

Once it has passed through the ion exchanger column the worked-up polyether solution is collected in a suitable product container. It is advisable to continuously monitor the pH of the outflowing solution to detect in good time when the ion exchanger capacity has been depleted. The operating conditions are preferably adjusted such that the outflowing product stream has a pH of not more than 7.

The thus obtained alkali-metal-free solution advantageously has a residual content of alkali metal based on the purified alkoxylation product of less than 10 ppm, preferably of less than 5 ppm.

In addition to the temperature, the type and amount of alcoholic solvent, the water and alkali metal content and also the chemical makeup and the usage amount of the ion exchanger, the quality of the purification in the fixed bed method is also influenced by the feed rate. The feed rate determines the average residence time of the solution in the fixed bed. In a preferred embodiment of the invention the process parameters are adapted to one other such that the pH of the effluxing product stream is not more than 7. It is preferable when the feed rate is throttled or the ion exchanger can be regenerated with acid when the pH of the product stream is greater than 7.

When a change of product is pending and before regeneration with acid it is preferable to free the ion exchanger fixed bed of product deposits by rinsing with solvent and/or water. It is advantageous to utilize the alcohol/water mixture used in step a) to wash out polyether residues.

After regeneration with acid it is necessary to rinse out acid residues with water and/or an organic solvent such as alcohol. The endpoint of the rinsing operation may be easily detected with the aid of a pH probe at the reactor outlet.

The feeding of the acid during the regeneration process may be effected either in the same flow direction as during supply of the alkaline alkoxylate solution (cocurrent process) or in the opposite direction (countercurrent process). The countercurrent process is preferred.

Preferred implementation of step c) of the method according to the invention:

The mixture resulting from step b) is freed of the solvent mixture in step c) of the method according to the invention. This is preferably achieved by distillative removal, in particular by vacuum distillation. If required final solvent residues may be removed from the polyether by stripping with water or an inert gas such as nitrogen. Removal of the solvent mixture may be performed either batchwise or else continuously and either in a stirred tank or, for example, in a thin film evaporator.

It is particularly advantageous to effect distillative removal of the first portion of the solvent mixture under atmospheric pressure and of the remainder under vacuum. Towards the end the temperature is preferably increased to over 100° C. and the pressure is preferably lowered to below 50 mbar until no more distillate flows. The alcohol/water distillate may be reused later for production of a solution as per step a).

The purified alkoxylation product obtained after the solvent removal is free of salts and does not generally require filtration. Nevertheless, a filtration may optionally be performed to remove any fine fractions of the ion exchanger.

In a preferred embodiment of the invention the processing of the alkali-catalysed alkoxylation product effects a reduction in the propenyl groups preferably resulting in a content of propenyl groups that is more than 40%, preferably 50% to 95%, lower compared to the alkoxylation product used for processing.

The present invention further provides an alkoxylation product obtainable by the method according to the invention as described hereinabove. Reference is made to the abovementioned preferred embodiments.

In the context of a preferred embodiment the alkoxylation product according to the invention has an acid number between 0 and 0.5 mg KOH/g, preferably not more than 0.3 mg KOH/g.

In a further preferred embodiment of the invention the alkoxylation product according to the invention is phosphate-free and the content of alkali metal, preferably sodium and potassium, is less than 10 ppm, preferably less than 5 ppm.

The products according to the invention are outstanding for the production of polyurethane foam, polymers such as polyether siloxanes and polyesters, as polyurethane foam stabilizers, for use in paints and for surface treatment, in coatings, adhesives and sealants, binders, cosmetic preparations, personal care products and cleaning products, as surfactants, emulsifiers, dispersants, defoamers, wetting agents, friction reducers, lubricants, glidants, release agents, additives in fuels such as petrol and diesel and rheology modifiers. In platinum-catalysed hydrosilylation reactions allyl polyethers especially show an excellent reactivity in the reaction with hydrosiloxanes even at Pt use concentrations as low as 2 ppm of Pt based on the reaction batch.

The invention therefore further provides for the use of the alkoxylation products according to the invention for producing polymers such as polyether siloxanes and polyester, as polyurethane foam stabilizers, in paints and for surface treatment, in coatings, adhesives and sealants, binders, cosmetic preparations, personal care products and cleaning products, as surfactants, emulsifiers, dispersants, defoamers, wetting agents, friction reducers, lubricants, glidants, release agents, additives in fuels such as petrol and diesel and rheology modifiers.

The invention further provides a PUR foam obtainable by reaction of at least one polyol component and at least one isocyanate component in the presence of a polyether siloxane obtained using the alkoxylation product according to the invention.

The examples presented below illustrate the present invention by way of example, without any intention of restricting the invention, the scope of application of which is apparent from the entirety of the description and the claims, to the embodiments specified in the examples. The method and the use according to the invention are described below by way of example, without any intention that the invention be limited to these illustrative embodiments.

### EXAMPLES

### GPC Measurements:

GPC measurements for determining the polydispersity and average molar masses Mw were conducted under the following measurement conditions: SDV 1000/10 000 A column combination (length 65 cm), temperature 30° C., THF as mobile phase, flow rate 1 ml/min, sample concentration 10 g/1, RI detector, evaluation against polypropylene glycol standard.

### Determination of the Content of Propenyl Polyethers:

The content of propenyl polyethers was determined using ¹H NMR spectroscopy. A Bruker Avance 400 NMR spectrometer was used. To this end, the samples were dissolved in deuteromethanol. The propenyl content is defined as the proportion of propenyl polyethers in mol % based on the entirety of all polyethers present in the sample.

Quantitative determination of the propionaldehyde content was effected using HPLC.

### Determination of the Alkali Metal Content in Polyethers:

Quantitative determination of the content of sodium and potassium was effected by digesting the samples with hot nitric acid and subjecting them to analysis by ICP-OES (inductively coupled plasma optical emission spectroscopy).

### Determination of the Iodine Number in Polyethers:

Iodine number determination was effected as per the Hanus titration method, known as method DGF C-V 17 a (53) of the Deutsche Gesellschaft für Fettwissenschaft.

### Determination of the Acid Number in Polyethers:

Acid number determination was performed as per a titration method based on DIN EN ISO 2114.

The processing procedures according to the invention used the following alkali-catalysed alkoxylation products (table 1):

| alkaline | | alkali | | propenyl | |
|---|---|---|---|---|---|
| polyether | chemical makeup | metal content | catalyst | content | iodine number |
| AP 1 | poly(oxypropylene) monobutyl | 3100 ppm | sodium | n/a | n/a |
| | ether | Na | methoxide | | |
| | Mw 700 g/mol, Mw/Mn 1.10 | | | | |
| AP 2 | poly(oxypropylene) monobutyl | 3300 ppm | potassium | n/a | n/a |
| | ether | K | methoxide | | |
| | Mw 1800 g/mol, Mw/Mn 1.16 | | | | |
| AP 3 | poly(oxyethylene)-co- | 1700 ppm | potassium | n/a | n/a |
| | (oxypropylene) monobutyl | K | methoxide | | |
| | ether | | | | |
| | Mw 1000 g/mol, Mw/Mn 1.08 | | | | |
| | 50 mol % EO, 50 mol % PO | | | | |
| AP 4 | poly(oxyethylene) monoallyl | 850 ppm | sodium | n/a | 64.0 g iodine/100 g |
| | ether | Na | methoxide | | |
| | Mw 400 g/mol, Mw/Mn 1.15 | | | | |
| AP 5 | poly(oxyethylene) monoallyl | 1600 ppm | potassium | 0.6 mol % | 43.0 g iodine/100 g |
| | ether | K | methoxide | | |
| | Mw 600 g/mol, Mw/Mn 1.10 | | | | |
| AP 6 | poly(oxyethylene)-co- | 1200 ppm | sodium | 1.1 mol % | 31.0 g iodine/100 g |
| | (oxypropylene) monoallyl ether | Na | methoxide | | |
| | Mw 900 g/mol, Mw/Mn 1.09 | | | | |
| | 70 mol % EO, 30 mol % PO | | | | |
| AP 7 | poly(oxyethylene)-co- | 1500 ppm | potassium | 20.3 mol % | 5.8 g iodine/100 g |
| | (oxypropylene) monoallyl ether | K | methoxide | | |
| | Mw 4400 g/mol, Mw/Mn 1.27 | | | | |
| | 50 mol % EO, 50 mol % PO | | | | |
| AP 8 | poly(oxyethylene)-co- | 1600 ppm | sodium | 1.3 mol % | 49.0 g iodine/100 g |
| | (oxypropylene) monoallyl ether | Na | methoxide | | |
| | Mw 500 g/mol, Mw/Mn 1.14 | | | | |
| | 60 mol % EO, 40 mol % PO | | | | |
| AP 9 | poly(oxyethylene)-co- | 4400 ppm | potassium | n/a | n/a |
| | (oxypropylene) glycol | K | hydroxide | | |
| | Mw 2800 g/mol, Mw/Mn 1.05 | | | | |
| | 55 mol % EO, 45 mol % PO | | | | |
| AP 10 | poly(oxyethylene)-co- | 2900 ppm | potassium | 5.1 mol % | 17.0 g iodine/100 g |
| | (oxypropylene) monoallyl ether | K | methoxide | | |
| | Mw 1500 g/mol, Mw/Mn 1.16 | | | | |
| | 10 mol % EO, 90 mol % PO | | | | |
| AP 11 | poly(oxyethylene)-co- | 2900 ppm | sodium | 4.6 mol % | 6.5 g iodine/100 g |
| | (oxypropylene) monoallyl ether | Na | methoxide | | |
| | Mw 4000 g/mol, Mw/Mn 1.28 | | | | |
| | 50 mol % EO, 50 mol % PO | | | | |

The following cation exchangers were employed, manufacturer data (table 2):

| | | particle size | capacity | water content | |
|---|---|---|---|---|---|
| Amberlyst ® | macroporous, | harmonic mean | ≥1.7 eq/l, | 52-57% | inventive |
| 15 | SO3H- | 0.60-0.85 mm | ≤4.7 eq/l | | |
| | functional | | | | |
| Amberlite ® | macroporous, | harmonic mean | >1.7 eq/l | 52-58% | inventive |
| 252H | SO3H- | 0.6-0.8 mm | | | |
| | functional | | | | |
| Lewatit ® | macroporous, | 0.315-1.6 mm | >4.3 eq/l | unknown | comparative |
| CNP-80 | COOH- | | | | example |
| | functional | | | | |

### Inventive Purification of the Alkaline Alkoxylation Products in a Stirred Reactor:

A temperature-controllable glass vessel fitted with a stirrer, temperature probe and pH meter was initially charged as per table 3 with 250 g of an alkaline polyether (see table 1), alcohol and water respectively and brought to the desired temperature with stirring. The pH meter indicated a pH of 12 to 14 in each case. Once the target temperature had been reached the respective amount of ion exchanger was added. A stopwatch was used to measure the time taken to achieve a pH of 7.

TABLE 3
Processing of alkaline alkoxylation products (250 g respectively) in a stirred reactor

| | | | | | | ion | | |
|---|---|---|---|---|---|---|---|---|
| | alkaline | | | solvent | Water | exchanger | temp. | time |
| experiment | polyether | ion exchanger | solvent | [g] | [g] | [g] | [° C.] | [min] |
| 1 | AP 2 | Amberlite ® 252H | ethanol | 250 | 10 | 25 | 45 | 35 |
| 2 | AP 2 | Amberlite ® 252H | ethanol | 125 | 10 | 25 | 45 | 45 |
| 3 | AP 2 | Amberlyst ® 15 | isopropanol | 250 | 10 | 25 | 45 | 22 |
| 4 | AP 2 | Amberlyst ® 15 | propanol | 250 | 10 | 25 | 45 | 30 |
| 5 | AP 2 | Amberlyst ® 15 | ethanol | 250 | 10 | 25 | 79 | 5 |
| 6 | AP 2 | Amberlite ® 252H | ethanol | 250 | 10 | 25 | 80 | 8 |
| 7 (noninventive) | AP 2 | Amberlite ® 252H | (none) | 0 | 10 | 25 | 80 | 60 |
| 8 | AP 2 | Amberlite ® 252H | ethanol | 75 | 10 | 25 | 80 | 20 |
| 9 (noninventive) | AP 2 | Lewatit ® CNP-80 | ethanol | 250 | 10 | 20 | 80 | >200 |
| 10 | AP 7 | Amberlite ® 252H | methanol | 250 | 10 | 25 | 45 | 35 |
| 11 | AP 7 | Amberlyst ® 15 | ethanol | 125 | 10 | 25 | 80 | 30 |
| 12 | AP 7 | Amberlyst ® 15 | ethanol | 250 | 10 | 25 | 80 | 25 |
| 13 | AP 7 | Amberlite ® 252H | isopropanol | 250 | 10 | 25 | 45 | 56 |
| 14 | AP 7 | Amberlite ® 252H | ethanol | 75 | 10 | 25 | 80 | 20 |
| 15 | AP 5 | Amberlite ® 252H | ethanol | 250 | 10 | 10 | 45 | 58 |
| 16 | AP 5 | Amberlite ® 252H | ethanol | 125 | 10 | 10 | 45 | 45 |
| 17 | AP 5 | Amberlite ® 252H | methanol | 250 | 10 | 10 | 45 | 25 |
| 18 | AP 5 | Amberlite ® 252H | ethanol | 125 | 10 | 10 | 80 | 7 |
| 19 | AP 3 | Amberlite ® 252H | ethanol | 75 | 10 | 20 | 80 | 20 |
| 20 | AP 3 | Amberlyst ® 15 | ethanol | 125 | 10 | 20 | 80 | 17 |
| 21 | AP 3 | Amberlite ® 252H | ethanol | 250 | 10 | 20 | 80 | 10 |
| 22 | AP 3 | Amberlyst ® 15 | ethanol | 250 | 10 | 20 | 80 | 10 |
| 23 | AP 6 | Amberlite ® 252H | ethanol | 75 | 10 | 15 | 80 | 26 |
| 24 | AP 6 | Amberlyst ® 15 | ethanol | 125 | 10 | 15 | 80 | 22 |
| 25 | AP 6 | Amberlyst ® 15 | propanol | 250 | 10 | 15 | 45 | 45 |
| 26 | AP 6 | Amberlite ® 252H | ethanol | 250 | 10 | 15 | 45 | 20 |
| 27 (noninventive) | AP 6 | Amberlite ® 252H | ethanol | 250 | 0 | 15 | 45 | >240 |
| 28 | AP 8 | Amberlyst ® 15 | ethanol | 250 | 10 | 15 | 45 | 18 |
| 29 | AP 9 | Amberlite ® 252H | ethanol | 250 | 10 | 25 | 45 | 45 |
| 30 | AP 9 | Amberlite ® 252H | ethanol | 125 | 10 | 25 | 45 | 45 |
| 31 | AP 4 | Amberlite ® 252H | ethanol | 250 | 10 | 10 | 45 | 20 |
| 32 | AP 4 | Amberlite ® 252H | ethanol | 125 | 10 | 10 | 45 | 20 |
| 33 | AP 10 | Amberlyst ® 15 | ethanol | 250 | 10 | 22.5 | 45 | 23 |
| 34 | AP 10 | Amberlyst ® 15 | ethanol | 125 | 10 | 22.5 | 45 | 48 |
| 35 (noninventive) | AP 10 | Amberlyst ® 15 | (none) | 0 | 10 | 22.5 | 45 | >210 |

The processed, neutralized polyether solutions were freed of alcohol and water by distillation and subsequently tested for alkali content and acid number. All poly ethers produced in accordance with the invention had a sodium/potassium content of <5 ppm and an acid number between 0 and 0.25 mg KOH/g. By contrast, experiments 9, 27 and 35 had to be aborted since pH 7 was not to be achieved even after several hours. The sample from experiment 7 was not analysed since a residence time of 60 min is uneconomic.

### Inventive Purification of the Alkaline Alkoxylation Products in a Fixed Bed Reactor:

An ion exchanger column fitted with a temperature probe and a heatable double shell and having an internal volume of approximately 600 ml was filled with 287 g of ion exchanger. A controllable piston pump was used to continuously supply, per experiment, 3-5 litres of the solutions, prepared as per table 4, of alkaline polyether (see table 1) in alcohol and water over an experimental duration of a number of hours. During the experimental duration the internal temperature was kept constant at the set target value by controlling the shell temperature. The residence time of the polyether solution in the column was varied via the feed rate of the pump. The pH of the product solution effluxing at the other end of the ion exchanger column was continually measured and in all cases indicated a pH of <7. The purified solutions were collected in a container and subsequently freed of the respective solvent. Alcohol and water were first removed by distillation at atmospheric pressure and then under vacuum at increasing temperatures up to 120° C. Clear, salt-free neutralized polyethers having an alkali metal content of <5 ppm and an acid number of 0 to 0.25 mg KOH/g were obtained.

TABLE 4
Processing of alkaline alkoxylation products in the fixed bed process,
usage amounts based on 2.5 kg of alkaline alkoxylation product

| | alkaline | | | solvent | water | feed | temp. |
|---|---|---|---|---|---|---|---|
| experiment | polyether | ion exchanger | solvent | [g] | [g] | [g/min] | [° C.] |
| A 1 | AP 1 | Amberlyst ® 15 | ethanol | 2500 | 100 | 12.9 | 47 |
| A 2 | AP 1 | Amberlite ® 252H | ethanol | 1250 | 100 | 15.3 | 80 |
| A 3 | AP 1 | Amberlite ® 252H | ethanol | 625 | 100 | 13.1 | 80 |
| A 4 | AP 2 | Amberlyst ® 15 | ethanol | 2500 | 100 | 16.0 | 45 |
| A 5 | AP 2 | Amberlite ® 252H | ethanol | 1250 | 100 | 16.0 | 78 |
| A 6 | AP 2 | Amberlite ® 252H | ethanol | 625 | 100 | 15.2 | 80 |
| A 7 | AP 3 | Amberlyst ® 15 | ethanol | 2500 | 100 | 13.2 | 45 |
| A 8 | AP 3 | Amberlyst ® 15 | isopropanol | 2500 | 100 | 13.9 | 45 |
| A 9 | AP 3 | Amberlite ® 252H | ethanol | 1250 | 100 | 16.5 | 79 |
| A 10 | AP 6 | Amberlyst ® 15 | ethanol | 2500 | 100 | 13.8 | 46 |
| A 11 | AP 6 | Amberlite ® 252H | ethanol | 1250 | 100 | 15.8 | 76 |
| A 12 | AP 7 | Amberlyst ® 15 | ethanol | 2500 | 100 | 15.1 | 45 |
| A 13 | AP 7 | Amberlite ® 252H | ethanol | 2500 | 100 | 11.8 | 78 |
| A 14 | AP 7 | Amberlite ® 252H | ethanol | 1250 | 100 | 13.4 | 78 |
| A 15 | AP 7 | Amberlite ® 252H | ethanol | 650 | 100 | 4.5 | 78 |
| A 16 | AP 8 | Amberlite ® 252H | ethanol | 2500 | 100 | 13.1 | 45 |
| A 17 | AP 8 | Amberlyst ® 15 | ethanol | 2500 | 100 | 14.2 | 45 |
| A 18 | AP 8 | Amberlite ® 252H | ethanol | 625 | 100 | 19.3 | 76 |
| A 19 | AP 4 | Amberlyst ® 15 | ethanol | 2500 | 100 | 14.4 | 46 |
| A 20 | AP 4 | Amberlite ® 252H | ethanol | 2500 | 100 | 15.6 | 79 |
| A 21 | AP 4 | Amberlite ® 252H | ethanol | 625 | 100 | 22.3 | 75 |
| A 22 | AP 5 | Amberlyst ® 15 | ethanol | 2500 | 100 | 13.4 | 45 |
| A 23 | AP 11 | Amberlite ® 252H | ethanol | 2500 | 100 | 17.7 | 80 |

As is shown in table 5 which follows, during passage through the ion exchanger fixed bed propenyl polyether and other odour-forming additions are efficaciously destroyed by hydrolysis and subsequently removed by distillation. The analytical results of the ¹H NMR spectra are confirmed by iodine number measurements which indicate a reduction in the content of double bonds compared to the respective alkaline starting polyether.

TABLE 5
Contents of double bonds, propenyl polyethers and other additions before and after inventive purification in a fixed bed reactor

| alkaline polyethers | | | | purified polyethers | | | |
|---|---|---|---|---|---|---|---|
| | iodine number | propenyl | | | iodine number | propenyl | |
| | [g iodine/100 | content [mol- | propionaldehyde | | [g iodine/100 | content [mol- | propionaldehyde |
| polyethers | g] | %] | [ppm] | experiment | g] | %] | [ppm] |
| AP 4 | 64.0 | n/a | not determined | A 19 | 64.0 | n/a | not determined |
| AP 4 | 64.0 | n/a | not determined | A 20 | 63.2 | n/a | not determined |
| AP 4 | 64.0 | n/a | not determined | A 21 | 63.8 | n/a | not determined |
| AP 5 | 43.0 | 0.6 | not determined | A 22 | 42.8 | 0.3 | not determined |
| AP 6 | 31.0 | 1.1 | 616 | A 11 | 30.8 | 0.4 | 6 |
| AP 7 | 5.8 | 20.3 | 2500 | A 15 | 5.3 | 3.7 | not determined |
| AP 7 | 5.8 | 20.3 | 2500 | A 14 | 5.3 | 3.9 | not determined |
| AP 7 | 5.8 | 20.3 | 2500 | A 12 | 4.8 | 2.4 | 370 |
| AP 8 | 49.0 | 1.3 | 940 | A 17 | 48.2 | 0.4 | not determined |
| AP 8 | 49.0 | 1.3 | 940 | A 18 | 48.4 | 0.6 | 17 |
| AP 2 | n/a | n/a | 1190 | A 6 | n/a | n/a | <160 |
| AP 1 | n/a | n/a | 180 | A 3 | n/a | n/a | 22 |
| AP 11 | 6.5 | 4.6 | not determined | A 23 | 6.4 | 2.1 | not determined |

The results in table 5 show clearly the reduction in the propenyl polyether proportions and the contents of propionaldehyde in the polyethers produced in accordance with the invention

1. A method of processing of alkali-catalyzed alkoxylation products using sulphonic acid-containing ion exchangers, comprising the steps of
a) providing a mixture comprising the alkali-catalyzed alkoxylation product to be processed, alcohol having 1 to 4 carbon atoms and water,
b) treating the mixture obtained from step a) with a sulphonic acid-containing cation exchanger at >40° C.,
c) removal of the alkoxylation product from the mixture obtained in step b).

2. The method according to claim 1 for removing alkali metal residues and odor-forming additions from the alkali-catalyzed alkoxylation products.

3. The method according to claim 1, wherein the alkali-catalyzed alkoxylation products to be processed originate from an alkali-metal-hydroxide- or alkali-metal-alkoxide-catalyzed alkoxylation process, have a molar mass Mw of 150 g/mol to 15 000 g/mol and a polydispersity Mw/Mn of 1.04 to 1.5.

4. The method according to claim 1, wherein the alkali-catalyzed alkoxylation products to be processed have 1 to 6 OH groups.

5. The method according to claim 1, wherein the ion exchanger used contains sulphonic acid groups, has an average particle size of 500-900 μm and has an ion exchange capacity of not less than 1.5 equ./litre (H⁺ Form).

6. The method according to claim 1, wherein in step b) the mixture for treatment with the ion exchanger is composed to an extent of 35 to 95 wt %, of alkoxylation product, to an extent of 4 to 64 wt %, of alcohol having 1 to 4 carbon atoms and to an extent of 1 to 15 wt % of water.

7. The method according to claim 1, wherein the alkali-catalyzed alkoxylation products to be processed are adducts of alkylene oxides selected from the group consisting of ethylene oxide, propylene oxide, 1-butylene oxide, 2-butylene oxide, isobutylene oxide and styrene oxide.

8. The method according to claim 1, wherein in step b) the mixture from step a) is passed through an ion exchanger bed at 45° C. to 100° C.

9. The method according to claim 1, wherein the processing of the alkali-catalyzed alkoxylation product effects a reduction in the content of propenyl groups that is more than 40%, lower compared to the alkoxylation product used for processing.

10. An alkoxylation product obtainable according to claim 1.

11. The alkoxylation product according to claim 10, wherein the acid number is between 0 and 0.5 mg KOH/g.

12. The alkoxylation product according to claim 10, wherein said product is phosphate-free and the content of sodium and potassium is less than 10 ppm.

13. (canceled)

14. A PUR foam obtainable by reaction of at least one polyol component and at least one isocyanate component in the presence of a polyether siloxane obtained using processed alkoxylation product according to claim 1.

15. The method according to claim 1, wherein the alkali-catalyzed alkoxylation products to be processed have 1 to 2 OH groups.

16. The method according to claim 1, wherein in step b) the mixture for treatment with the ion exchanger is composed to an extent of 45 to 85 wt % of alkoxylation product, to an extent of 12 to 53 wt % of alcohol having 1 to 4 carbon atoms, and to an extent of 2 to 10 wt % of water.

17. The method according to claim 1, wherein in step b) the mixture for treatment with the ion exchanger is composed to an extent of 50 to 80 wt % of alkoxylation product, to an extent of 17 to 48 wt % of alcohol having 1 to 4 carbon atoms, and to an extent of 3 to 8 wt % of water.

18. The alkoxylation product according to claim 10, wherein the acid number is not more than 0.3 mg KOH/g.

19. The alkoxylation product according to claim 10, wherein said product is phosphate-free and the content of sodium and potassium is less than 5 ppm.

20. The method according to claim 1, wherein the processing of the alkali-catalyzed alkoxylation product effects a reduction in a content of propenyl groups that is from 50% to 95%, lower compared to the alkoxylation product used for processing.

21. The method according to claim 1, wherein the alkali-catalyzed alkoxylation products to be processed are adducts of alkylene oxides, selected from the group consisting of ethylene oxide and propylene oxide.
